(19) United States
(12) Patent Application Publication
(10) Pub. No.: US 2003/0036049 A1
(43) Pub. Date: Feb. 20, 2003

(54) Optical method for testing sensitivity of cells

(75) Inventors: Samario Chaitchik, Tel Aviv (IL); Yael S. Schiffenbauer, Rosh Haain (IL); Ella Trubniyakov, Ramla (IL)

Correspondence Address: DR. MARK FRIEDMAN, LTD., C/O BILL POLKINGHORN- DISCOVERY DISPATCH, 9003 FLORIN WAY, UPPER MARLBORO, MD, 20772, US

(73) Assignee: MEDIS EL LTD.

(21) Appl. No.: 09/752,453

(22) Filed: Jan. 3, 2001

(51) Int. Cl. C12Q 1/00; G06F 19/00; G01N 33/48; G01N 33/50

(52) U.S. Cl. 435/4; 702/19

(57) ABSTRACT

According to the present invention there is provided a method of cell selection and characterization that is related to a field known as cytometry. Further, the present invention relates to a method for testing human cancer cells sensitivity (or alternatively, cells resistance) to anti-cancer drugs, by using laser-based non-flow cytometry.

### FIELD AND BACKGROUND OF THE INVENTION

The present invention is an optical method for testing sensitivity of cells. The method relies upon cytometry for testing drug sensitivity of cells, i.e. their survival rate after exposure to chemical or biological agents.

A major clinical problem in the area of oncology is that cancers classified as identical according to their histopathological characteristics are highly individual in their drug sensitivities. Current treatment is often based on the results of randomized trials performed on patients who have similar types and stages of disease. Unfortunately, these treatment protocols have shown that only a limited number of patients respond as expected.

Several different chemosensitivity and chemoresistance assays have been developed through the years in order to select potentially more effective chemotherapy regimens and to avoid the toxicity of potentially ineffective drugs. Hamburger and Salmon developed the clonogenic assay, based on the unique ability of transformed cells to grow on soft agar and subsequently form colonies (Hamburger, A. W., and Salmon, S. E., Science 197: 461, 1976). Modifications of the clonogenic assay followed, which assured improved assessability rate (number of tumors which can be successfully cultured) and shorter assays, using radioactive-labeling [³H] of the nucleic acid precursor thymidine (Tanigawa, N. et al, Cancer Res. 42: 2159-2164, 1982; Kern, D. H. et al., Ibid. 45:5436-5441, 1985). Novel techniques were also developed such as the dye exclusion (Weisenthal, L. M. et al., Ibid. 43: 749-757, 1983) and the gel-supported primary culture assay (Vescio, R. A. et al., Proc. Natl. Acad. Sci. USA 84: 5029-5033, 1987) followed by the commercially available fluorescent cytoprint (Rotman, B. et al., In vitro Cell. Develop. Biol. 24: 1137-1146, 1988) and extreme drug resistance tests (Kern, D. H. and Weisenthal, L. M., J. Natl. Cancer Inst. 82: 582-588, 1990). Another method for testing chemosensitivity of cancer cells is described in U.S. Pat. No. 5,356,793.

A significant shortcoming of the aforementioned prior art is the long incubation time required for the formation of colonies. In many cases, culturing of tumor cells is impossible.

Further, recent scientific literature suggests that there have been no clinical trials showing that either of these assays is associated with improved patient survival (Brown, E. and Markman, M., Cancer 77:1020-1025, 1996).

The CellScan is a laser-based, non-flow cytometer equipped with a unique cell carrier grid that contains 10,000 wells, each of which can accommodate a single cell (Deutch, M. and Weinreb, A., Cytometry 16: 214-226, 1994). It is based on a novel technology described by U.S. Pat. Nos. 4,729,949, 4,772,540, 5,272,081, 5,310,674 and 5,506,141. By using this technology, fluorescence intensity and polarization of individual cells in a heterogeneous cell population can be rapidly and repeatedly monitored providing important information on specific cell responses to external stimuli over time. The CellScan is the only automated cytometry that enable characterization of single non-adherent cells being located at identifiable positions.

There is thus a widely recognized need for, and it would be highly advantageous to have, a method for testing human cancer cells sensitivity to anti-cancer drugs devoid of the above limitations.

### SUMMARY OF THE INVENTION

According to the present invention there is provided a method for testing drug sensitivity (or alternatively, drug resistance) of cells with respect to a drug. The method includes the steps of: preparing a suspension of the cells in a liquid; exposing a portion of the cells to a drug; causing the cells to reside individually in defined locations, adding at least one substance capable of imparting a measurable degree of fluorescence to the cells in the suspension; such that each individual cell corresponds to exactly one of the defined locations, and such that the defined locations can be individually accessed by an assay device; and assaying by means of the assay device at least a portion of the cells in the defined locations at least one time as a means of determining the drug sensitivity (or alternatively, drug resistance) thereof.

According to further features in preferred embodiments of the invention described below, the drug is encapsulated in any item selected from the group consisting of a virus, a micelle and a liposome.

According to still further features in the described preferred embodiments, the drug is a nucleic acid.

According to still further features in the described preferred embodiments, the cells are incubated in culture dishes and harvested thereafter.

According to still further features in the described preferred embodiments, the cells are harvested with a proteolytic enzyme.

According to still further features in the described preferred embodiments, the proteolytic enzyme is trypsin.

According to still further features in the described preferred embodiments the cells are washed at least once.

According to still further features in the described preferred embodiments the cells are incubated with the substance capable of imparting a measurable degree of fluorescence.

According to still further features in the described preferred embodiments the substance capable of imparting a measurable degree of fluorescence is selected from the group consisting of: a substance that differentially stains mitochondria of living cells; a precursor of fluorescent substance that differentially stains living cells; and a fluorophore that stains nucleic acids.

According to still further features in the described preferred embodiments the method further includes the step of reporting results from the assaying.

According to still further features in the described preferred embodiments the method further includes the step of: processing the results to give at least one item selected from the group consisting of: a histogram of fluorescence intensity versus number of cells; a histogram of fluorescence polarization versus number of cells; a histogram of fluorescence intensity versus time; a histogram of fluorescence polarization versus time; a scatter plot of fluorescence intensity at one wavelength range versus fluorescence intensity at another wavelength range; a scatter plot of fluorescence intensity versus fluorescence polarization; a scatter plot of fluorescence polarization versus fluorescence intensity; average fluorescence intensity; standard deviation of fluorescence intensity; standard error of fluorescence intensity; average fluorescence polarization; standard deviation of fluorescence polarization; and standard error of fluorescence polarization.

According to still further features in the described preferred the histograms include error bars.

According to still further features in the described preferred embodiments the step of processing further comprises at least one sub-step selected from the group consisting of: presenting at least one of the at least one item on a computer screen; printing at least one of the at least one item on a printer; plotting at least one of the at least one item on a plotter; and storing at least one of the at least one item on a data-storage device.

### BRIEF DESCRIPTION OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 is a flow chart describing a method for testing sensitivity of cells to chemical treatment;

FIG. 2 contains bar plots describing the results of measuring dose-dependent cell viability of sensitive and resistant cells after chemical treatment by counting viable cells in a heamocytometer after excluding dead cells by trypan blue staining;

FIG. 3 contains bar plots describing dose-dependent results of measuring rhodamine 123 (Rh123) fluorescence intensity imparted from sensitive and resistant cells after chemical treatment;

FIG. 4 contains charts describing Rh123 stained sensitive and resistant cell populations characterized by fluorescence intensity (FI, vertical axis) and fluorescence polarization (FP, horizontal axis);

FIG. 5 contains histograms describing the results of fluorescence polarization measurements performed on cells stained with fluorescein diacetate (FDA), following treatment with no drug (dark grey columns), with low drug dose (light grey columns) and with high drug dose (black columns) in sensitive and resistant cells;

FIG. 6 contains charts describing cell populations stained with acridine orange (AO) following drug treatment of sensitive and resistant cells, characterized by fluorescence intensity (FI, vertical axis) and fluorescence polarization (FP, horizontal axis).

### DETAILED DESCRIPTION OF THE INVENTION

The present invention is of an optical method for testing sensitivity (or alternatively, resistance) of cells to chemical and biological treatment which can be used to accurately assay individual cells.

Specifically, the present invention can be used to increase accuracy of predictions regarding the response of cells derived from a tumor sample to a therapeutic agent.

The principles and operation of the method for testing sensitivity of cells to chemical and biological treatment according to the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting. According the present invention there is provided a method 10 for testing drug sensitivity of cells with respect to a drug. The method 10 includes the steps of: suspending 50 the cells in a liquid. The liquid may be, for example, a saline solution, a buffered saline solution, or a cell culture media. The method further includes the step of exposing 70 at least a portion of the cells in suspension to a drug and adding 80 at least one substance capable of imparting a measurable degree of fluorescence. Further included in the method is the step of causing the cells to reside individually in defined locations 90. This is done so that each individual cell corresponds to exactly one of the defined locations, and so that the defined locations can be individually accessed by an assay device. One ordinarily skilled in the art will be able to incorporate commercially available assay devices for use with the method of the present invention. One example of such an assay device is the CellScan™ (Medis-El, Yehud, Israel) although other devices might be employed without significantly altering performance of the method. The method further includes the step of assaying 110 by means of the assay device at least a portion of the cells in the defined locations at least one time as a means of determining their drug sensitivity.

The drug may be, for example encapsulated in any item selected from the group consisting of a virus, a micelle and a liposome. According to some embodiments of the invention, the drug is a nucleic acid. In some cases, the cells are incubated in culture dishes and harvested thereafter. Harvesting 40 of the cells may be, for example, with a proteolytic enzyme, for example trypsin or any other commercially available enzyme commonly employed for cell harvest in tissue culture. Typically, but not always, the cells are washed at least once.

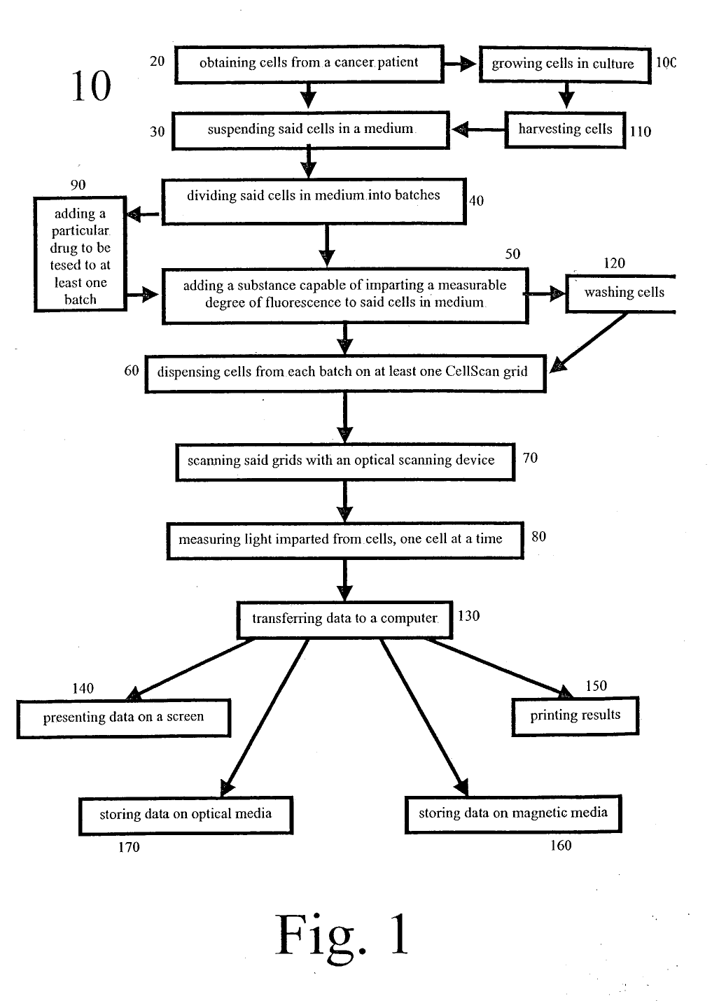

According to method 10 (FIG. 1) cells are incubated with the substance capable of imparting a measurable degree of fluorescence. The substance capable of imparting a measurable degree of fluorescence may be, for example, a substance that differentially stains mitochondria of living cells, a precursor of a fluorescent substance that differentially stains living cells or a fluorophore that stains nucleic acids.

Method 10 may further include the step of: reporting results from the assaying.

Method 10 may further include the step of: processing the results. This processing may produce, for example, a histogram of fluorescence intensity versus number of cells, a histogram of fluorescence polarization versus number of cells, a histogram of fluorescence intensity versus time, a histogram of fluorescence polarization versus time, a scatter plot of fluorescence intensity versus fluorescence polarization, a scatter plot of fluorescence polarization versus fluorescence intensity, average fluorescence intensity, standard deviation of fluorescence intensity, standard error of fluorescence intensity, average fluorescence polarization, standard deviation of fluorescence polarization and standard error of fluorescence polarization. These histograms may include error bars.

Processing may further include sub-steps including, but not limited to, presenting 130 at least one of the at least one item on a computer screen, printing 140 at least one of the at least one item on a printer, plotting at least one of the at least one item on a plotter and storing at least one of the at least one item on a data-storage device 150, 160. The light imparted from each cell is measured 110.

The cultured cells can be washed at any time with any kind of buffer, such as PBS, the PBS may be supplemented with divalent cations, such as calcium or magnesium. One ordinarily skilled in the art will be able to choose another suitable washing solution, for example a buffered saline solution, or a buffered saline solution including an appropriate concentration of a suitable detergent.

The fluorescence may be imparted after the substance undergoes a chemical change inside living cells, or after its binding to a specific target inside cultured cells, such as nucleic acids or mitochondria of living cells.

According to alternate preferred embodiments, cultured cells may be shaken or stirred during incubation to give them continuous exposure to the drug and to the substance capable of imparting a measurable degree of fluorescence.

In some cases the method includes presenting at least one of the diagrams or statistical parameters on a computer screen 130.

According to still further features in the described preferred embodiments, at least one of the diagrams and the statistical parameters may be printed 140. At least one of the diagrams and the statistical parameters may be plotted as a diagram or stored on any media such as magnetic media 150. For purposes of this specification and the accompanying claims, the phrase “magnetic media” includes but is not limited to floppy discs, zip discs and jazz disks, and optical media 160, for example a compact disc.

Cancer cells are obtained from a patient by methods known to one ordinarily skilled in the art such as biopsy of solid tumor or taking blood samples.

For purposes of this specification and the accompanying claims, the term ‘drug’ refers to any substance or pharmaceutical composition known to one ordinarily skilled in the art as being of potential use in chemotherapy of cancer patients. Further included in the definition of drug are materials which are being assayed for potential use in treatment, although they are of no known efficacy.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

The present invention successfully addresses the shortcomings of the presently known configurations by providing a fast, convenient and automated method for allowing statistical analysis of the sensitivity of malignant tumor cells to one or more drugs.

Materials and Experimental Methods

The following materials and methods are provided in conjunction with the specific examples hereinbelow.

Cell culture: T80 and T47D human breast cancer cells were routinely cultured in DMEM supplemented with 10% fetal calf serum (FCS) (Biological Industries, Beit Haemek, Israel), 4 mM glutamine (Biological Ind.), 100 u/ml penicillin, 0.1 milligram/milliliter streptomycin, 12.5 units/milliliter nystatin (Biological Ind.) and 0.2 units/milliliter insulin (Lilly, Fegersheim, France).

About 2×10⁵ cells were plated on 6 well-plates and left overnight to adhere. Two different concentrations of different drugs were added for a period of 24, 48 and 72 hours and then cells were harvested with trypsin, centrifuged, and resuspended in culture medium until staining.

Cell staining and loading: Three samples were prepared for each treatment at a specific time point and stained each with a different dye: fluorescein diacetate (FDA), rhodamine 123 (Rh123), and acridine orange (AO).

FDA is a non-fluorescent molecule which gets accumulated and hydrolyzed inside living cells to yield the fluorescent molecule fluorescein by the process termed fluorochromasia (Rotman, B. and Papermaster, B. W., Biochemistry 55: 134-141, 1966).

Cells in suspension were stained for 5 minutes with FDA at a final concentration of 2.4 micromolar. The cells were then loaded on the cell carrier, washed twice with PBS supplemented with Mg⁺² and Ca⁺², and fluorescence intensity and polarization were measured by an He/Cd laser-based CellScan™ electro-optical scanner.

Rh123 is a lipophilic cationic fluorochrome that selectively accumulates in the mitochondria of living cells and whose specific uptake or release depend upon mitochondrial membrane potential (Johnson, L. V. et al., Proc. Natl. Acad. Sci. 77: 990-994, 1980; Johnson, L. V. et al., J Cell. Biol., 88: 526-535, 1981). Dissipation of the membrane potential was recently associated with early stages of apoptosis (Cossarizza, A. et al., Exp. Cell. Res. 214: 323-330, 1994; Lizard, G. et al., Cytometry 21: 275-283, 1995; Petit, P. X. et al. J. Cell Biol. 130: 157-165, 1995).

Rh123 (1 microgram/milliliter final concentration) was added to cells suspended in culture medium for 30 minutes at 37° C. Then the cells were washed twice with cold PBS supplemented with Mg⁺² and Ca⁺², loaded on the cell carrier and fluorescence intensity and polarization were measured by an Argon laser-based Electro-optical scanner.

AO is usually used as a direct probe for nucleic acids in fixed or permeabilized cells. When used under appropriate conditions of concentration and pH it gives bright green fluorescence when bound to double-stranded DNA and red deep fluorescence when bound to single strand RNA and can serve for estimation of cellular DNA content and transcriptional activity. AO was added directly to the cell carrier loaded with cells for 5 minutes at a final concentration of 10 microgram/milliliter, washed twice directly on the grid and measured on an Argon laser-based Electro-optical scanner.

Electro-optical scanner measurements: Following staining (for Rh123 and FDA) or prior staining (for AO), 90 microliters of a cell suspension of 2×10⁶ cells/ml were loaded on the cell carrier grid described in detail by Deutch, M. and Weinreb, A. (Cytometry 16: 214-226, 1994). This grid is described by U.S. Pat. Nos. 4,729,949, 4,772,540, 5,272,081, 5,310,674 and 5,506,141. Briefly, the cell carrier grid consists of an array of 100×100 conical wells mounted on a holder, which contains a reservoir filled with buffer. Mild negative pressure is applied resulting in the trapping of a single cell per well. Following this procedure the cells can be washed or restained without changing their location.

For the excitation of FDA-stained cells an He/Cd laser was utilized (442 nm, excitation power of 4.5 microwatt) and emission measured at 527 nm. For the detection of AO and Rh123-stained cells an Argon laser was utilized (488 nm, excitation power of 15 microwatt) and emission measured at 530 nm.

The fluorescence emission of an area containing 20×20 wells was measured using a preset photon counting mode in which the same number of photons is counted for each cell (10,000), canceling differences in precision from strong and weak-emitting cells. The fluorescence intensity (FI) and polarization (FP) for each cell was determined online.

Data processing and analysis: The polarization and intensity values presented are an average of the values measured for each cell in the scanned area. Two consecutive measurements of the same sample were performed using the standard deviation as the experimental error.

### EXAMPLES

Reference is now made to the following examples, which together with the above descriptions, illustrate the invention in a non limiting fashion.

The inventors have employed two human breast cancer cell lines as an experimental model to establish the changes that can be observed by scanning the cells after positioning them on a grid (e.g. CellScan™ grid, Medis-El, Yehud, Israel). The results can be correlated to drug efficacy, prior to applying such measurements to tissue biopsies from solid human tumors.

Generally, the nomenclature used herein and the laboratory procedures utilized in the present invention include medical and cellular biology techniques. Such techniques are thoroughly explained in the literature. See, for example, methodologies as set forth in:

“Cell Biology: A Laboratory Handbook” Volumes I-III, by Cellis, J. E., ed. (1994); “Culture of Animal Cells—A Manual of Basic Technique”, by Freshney, R. I., ed. (2000); “Animal Cell Culture”, by Freshney, R. I., ed. (1992).

All of which are incorporated by reference as if fully set forth herein. Other general references are provided throughout this document. The procedures therein are well known in the art and are provided for the convenience of the reader. All the information contained therein is incorporated herein by reference.

### Example 1

### Cell Staining and Loading

The purpose of cells staining is preparing them for fluorescence measurments, therefore cells were stained with dyes capable of imparting a measurable degree of fluorescence. The results presented below are for experiments in which Rh123, FDA or AO were used for staining of cells, although other fluorochromes were used in other experiments not presented here.

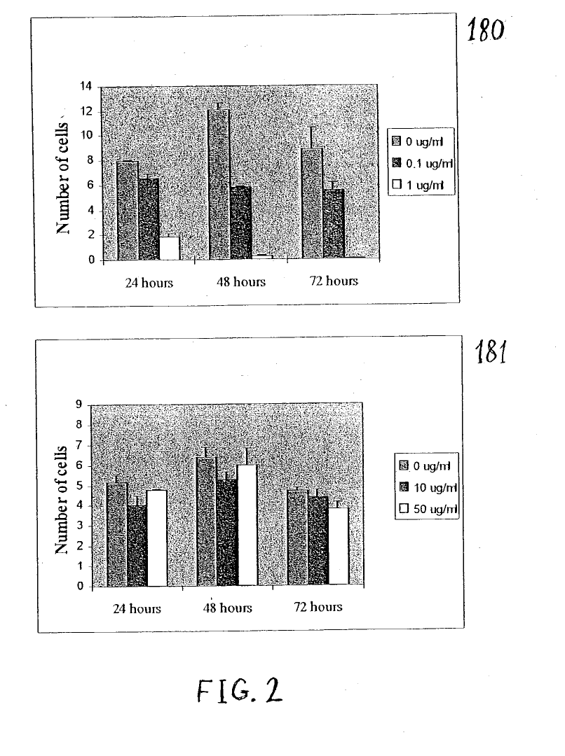

Navelbine and 5-Fluorouracil (5FU) where added at low and high doses to T80 and T47D cells respectively for 24, 48, and 72 hours. The cells were counted on a hemocytometer, using trypan blue for exclusion of dead cells, for the purpose of Electro-optical scanner -independent estimation of drug efficacy. The results are presented in FIG. 2. Panel 180 shows the effect of Navelbine on sensitive cells (T80). Panel 181 shows no effect of 5-Fluorouracil (5FU) on resistant cells (T47). The cell count revealed a cytotoxic effect of Navelbine on T80 cells already 24 hours after starting treatment in all concentrations used (panel 180). On the other hand, T47D cells seem to be resistant to 5FU as appreciated from the mild and not significant effects of the drug on the number of cells counted following drug exposure (panel 181).

### Example 2

### Electro-optical Scanner Measurements, Data Processing and Analysis

The purpose of this measurement is tracing changes in fluorescence imparted from single cells over time. In an attempt to correlate the sensitivity or resistance of the cells to the drugs as observed by cell counting with Electro-optical scanner parameters, the inventors stained the cells with different fluorochromes and measured the fluorescence intensity (FI) and fluorescence polarization (FP) of control and treated cells at different time points.

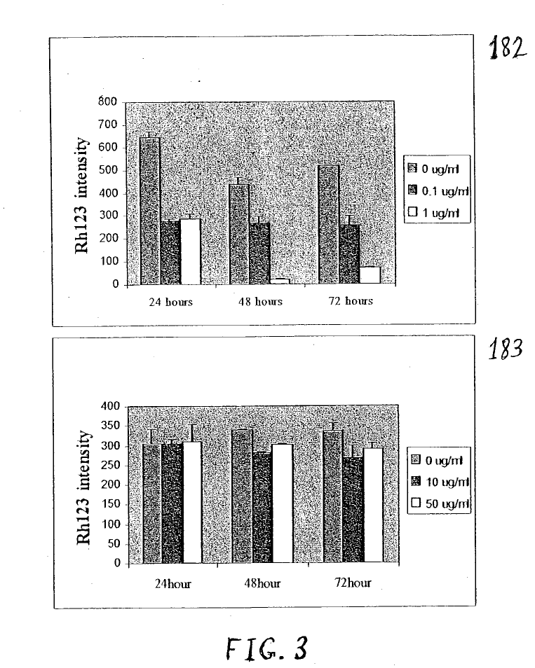

A comparison between Navelbine sensitive cells (T80) and 5FU resistant cells (T47D) implicated from Rh123 fluorescence measurements is presented in FIG. 3. The decrease in Rh123 fluorescence intensity is obvious and markedly significant in T80 cells treated with Navelbine as compared to control untreated cells (panel 182). On the other hand, in T47D cells, which showed resistance against 5FU treatment, no significant change in fluorescence intensity was observed (panel 183). A very good correlation was found between the degree of growth inhibition estimated from the number of cells counted, and the decrease in fluorescence intensity as measured for the Rh123 stained cells.

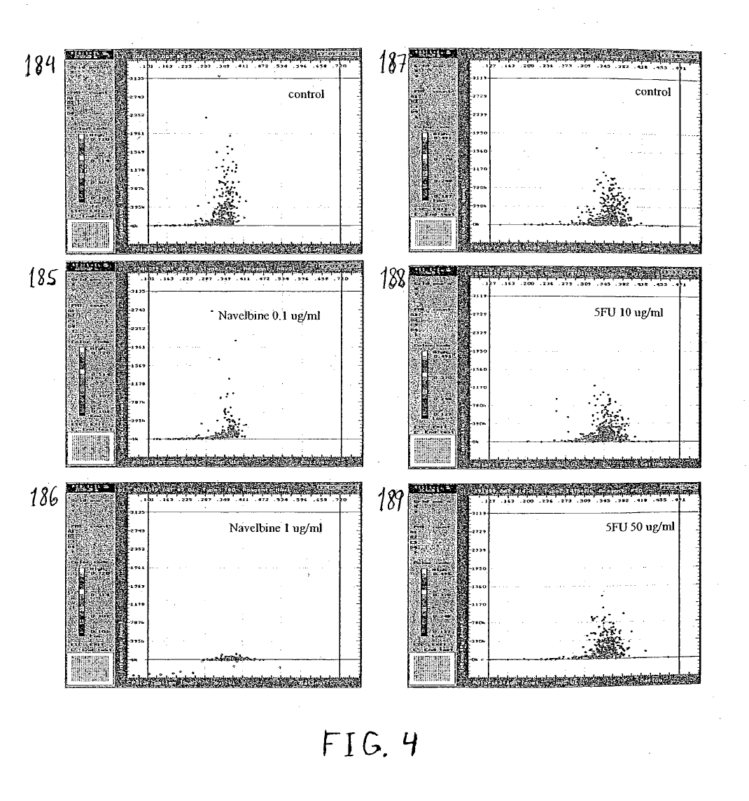

FIG. 4 shows the results of Rh123 fluorescence as measured one-cell-at-a-time. The decrease of mean fluorescence intensity following drug exposure of the Rh123 stained Navelbine-sensitive T80 cells was clear one day after starting treatment (panels 184-186), as compared to no significant change of mean fluorescence intensity following drug exposure of the Rh123 stained 5FU-resistant T47D cells (panels 187-189).

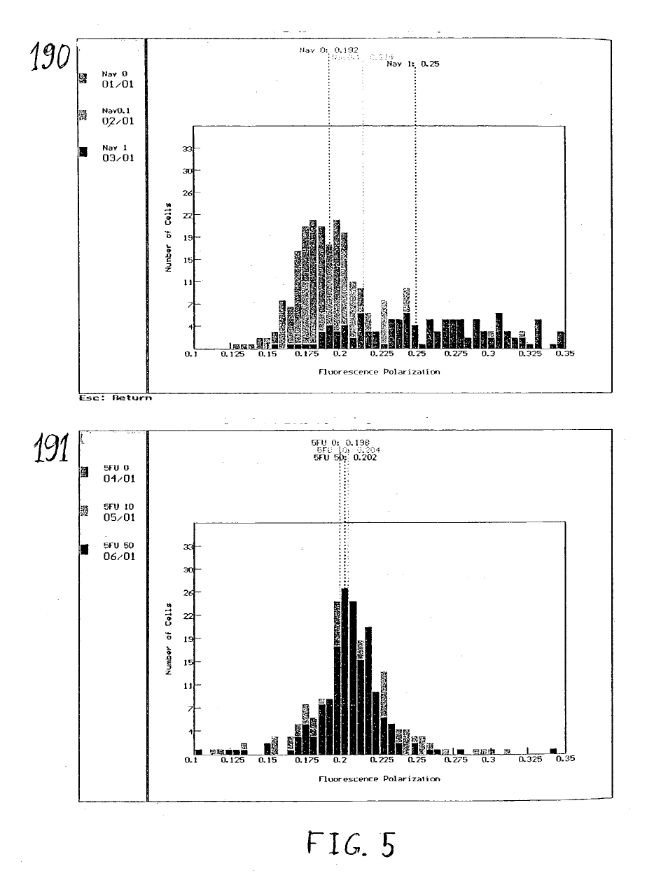

The increase of mean polarization of FDA fluorescence imparted from stained Navelbine-sensitive T80 cells following drug exposure was clearly observed by the Electro-optical scanner (FIG. 5, panel 190). The increase of mean polarization is significant as compared to no significant change of mean fluorescence polarization following drug exposure of the FDA-stained 5FU-resistant T47D cells (FIG. 5, panel 191).

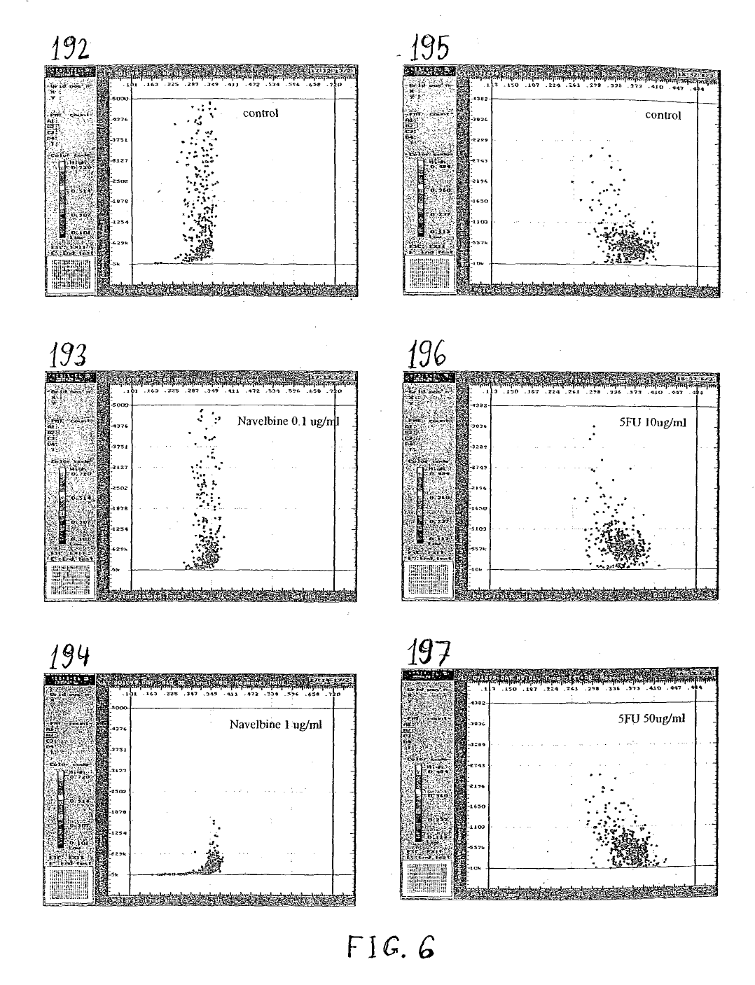

FIG. 6 shows the results of AO fluorescence as measured one-cell-at-a-time. The decrease of mean fluorescence intensity following drug exposure of the AO stained Navelbine-sensitive T80 cells was clear one day after starting treatment (panels 192-194), as compared to no significant change of mean fluorescence intensity following drug exposure of the AO stained 5FU-resistant T47D cells (panels 195-197).

These results show the significance of using Electro-optical scanner grid for measurements of fluorescence emitted from single cells as a reliable method for testing sensitivity (or alternatively, resistance) of cells to chemical and biological treatments.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

What is claimed is:

1. A method of testing drug sensitivity of cells with respect to a drug, the method comprising the steps of:
(a) preparing a suspension of the cells in a liquid;
(b) exposing a portion of the cells to the drug;
(c) adding at least one substance capable of imparting a measurable degree of fluoescence to the cells in said suspension;
(d) causing the cells to reside individually in defined locations, such that each individual cell corresponds to exactly one of said defined locations, and such that said defined locations can be individually accessed by an assay device;
(e) assaying by means of said assay device at least a portion of the cells in said defined locations at least one time as a means of determining the drug sensitivity thereof.

2. The method of claim 1, wherein the drug is encapsulated in any item selected from the group consisting of a virus, a micelle and a liposome.

3. The method of claim 2, wherein the drug is a nucleic acid.

4. The method of claim 1, wherein the cells are incubated in culture dishes and harvested thereafter.

5. The method of claim 4, wherein cells are harvested with a proteolytic enzyme.

6. The method of claim 5, wherein said proteolytic enzyme is trypsin.

7. The method of claim 1, wherein the cells are washed at least once.

8. The method of claim 1, wherein cells are incubated with said substance capable of imparting a measurable degree of fluorescence.

9. The method of claim 8, wherein said substance capable of imparting a measurable degree of fluorescence is selected from the group consisting of:
(a) a substance that differentially stains living cells;
(b) a precursor of fluorescent substance that differentially stains living cells; and
(c) a fluorophore that stains nucleic acids.

10. The method of claim 1, further including the step of:
(f) reporting results from said assaying.

11. The method of claim 10, further including the step of:
(g) processing said results to give at least one item selected from the group consisting of:
(i) a histogram of number of cells versus fluorescence intensity;
(ii) a histogram of number of cells versus fluorescence polarization;
(iii) a histogram of fluorescence intensity versus time;
(iv) a histogram of fluorescence polarization versus time;
(v) a scatter plot of fluorescence intensity versus fluorescence polarization;
(vi) a scatter plot of fluorescence polarization versus fluorescence intensity;
(vii) average fluorescence intensity;
(viii) standard deviation of fluorescence intensity;
(ix) standard error of fluorescence intensity;
(x) average fluorescence polarization;
(xi) standard deviation of fluorescence polarization; and
(xii) standard error of fluorescence polarization.

12. The method of claim 11, wherein said histograms include error bars.

13. The method of claim 11, wherein said step of processing further comprises at least one sub-step selected from the group consisting of:
(1) presenting at least one of said at least one item on a computer screen;
(2) printing at least one of said at least one item on a printer;
(3) plotting at least one of said at least one item on a plotter; and
(4) storing at least one of said at least one item on a data-storage device.
